(19) United States
(12) Patent Application Publication
(10) Pub. No.: US 2010/0284962 A1
(43) Pub. Date: Nov. 11, 2010

(54) MODIFIED TUMOR NECROSIS FACTOR-BETA

(75) Inventor: Mike A. Clark, Cheyenne, WY (US)

Correspondence Address: FISH & RICHARDSON P.C., PO BOX 1022, MINNEAPOLIS, MN, 55440-1022, US

(73) Assignee: OncoPharmacologics, Inc., Cheyenne, WY (US)

(21) Appl. No.: 12/436,440

(22) Filed: May 6, 2009

(51) Int. Cl. A61K 38/19; C07K 14/525; A61P 35/00

(52) U.S. Cl. 424/85.1; 530/351

(57) ABSTRACT

Compositions including modified TNF-beta polypeptides and methods of using the compositions are disclosed herein.

### TECHNICAL FIELD

The present invention is directed to compositions that include a modified cytokine and to methods of using the compositions, and more particularly, to modified tumor necrosis factor (TNF)-beta polypeptides and methods of using modified TNF-beta polypeptides to treat cancer or symptoms of a cancer.

### BACKGROUND

Tumor necrosis factor-beta (TNF-beta, also known as lymphotoxin alpha, or LTA, and TNF-β) is a soluble cytokine secreted by lymphocytes. TNF-beta is a member of the tumor necrosis factor cytokine family. The amino acid sequence of human TNF-beta is 33% identical to the sequence of human tumor necrosis factor-alpha (TNF-alpha; Pennica et al., Nature 312:724-729, 1984). TNF-beta is cytotoxic to tumor cells in vitro and also mediates proinflammatory and antiviral effects (Gray et al., Nature, 312:721-724, 1984; Shalaby et al., J. Immunol., 135:2069-2073, 1985; Wong et al., Nature, 323:819-822, 1986).

TNF-beta and TNF-alpha have similar overall structures and exert similar biological activities. However, TNF-beta has less potent proinflammatory activities in some contexts (Chaturvedi et a., J. Biol. Chem., 269(20):14575-14583, 1994). TNF-alpha has been tested in clinical studies of tumor therapy, which showed that clinical use of TNF-alpha is limited by its toxicity. The maximum tolerated dose of systemically administered TNF-alpha is 8-20-fold less than the efficacious dose in animals (Spriggs and Yates, Cancer chemotherapy: experiences with TNF administration in humans, Beutler B. eds., Tumor Necrosis Factors: The Molecules and Their Emerging Role in Medicine, 383-406, Raven Press New York 1992). Dose limiting toxicities of TNF-alpha include hypotension, fever, and liver toxicity (Lejeune et al., Cancer Immun., 6:1-17, 2006). In addition, systemic therapy with TNF-alpha is limited by its short circulating half life (<20 minutes in humans). TNF-alpha is used successfully in isolated limb perfusion regimens for treating a limited subset of tumors such as soft tissue sarcoma and melanoma metastases confined to the limb (van Horssen et al., Oncologist, 11:397-408, 2006).

### SUMMARY

The present invention provides TNF-beta polypeptides that are modified so as to increase their circulating half life and efficacy in vivo. The modifications include amino acid sequence mutations that permit attachment of molecules that increase half life while preserving biological activity. Also provided are nucleic acids encoding the modified TNF-beta polypeptides, host cells for expression of the polypeptides, compositions including the polypeptides (e.g., wherein the polypeptides are conjugated to a polymer such as polyethylene glycol (PEG)), and methods of treating diseases with the compositions.

In one aspect, this document features a tumor necrosis factor-beta (TNF-beta) polypeptide, wherein the TNF-beta polypeptide is modified to introduce one or more pegylation sites within amino acids 1-20 of SEQ ID NO:1. The TNF-beta polypeptide can be modified to introduce two or more pegylation sites within amino acids 1-20 of SEQ ID NO:1 (e.g., wherein the TNF-beta polypeptide is modified to introduce 2, 3, or 4 pegylation sites, e.g., wherein the TNF-beta polypeptide is modified to introduce one or more lysines). The TNF-beta polypeptide can be modified to include a lysine at one or both of positions 2 and 10 of SEQ ID NO:1. The lysine at one or both of positions 2 and 10 of SEQ ID NO:1 can be introduced by substitution. The TNF-beta polypeptide can be further modified to eliminate one or more pegylation sites. For example, the TNF-beta polypeptide can be modified to eliminate one or more lysine residues within amino acids 21-171 of SEQ ID NO:1 (e.g., at position 28 of SEQ ID NO:1). The one or more lysines can be eliminated by substitution with a glutamic acid, valine, aspartic acid, alanine, isoleucine, or leucine. The TNF-beta polypeptide can have at least 95% identity to SEQ ID NO:1. The TNF-beta polypeptide can have the amino acid sequence of SEQ ID NO:2.

In another aspect, this document features a TNF-beta polypeptide, wherein the TNF-beta polypeptide is modified to eliminate one or more pegylation sites within amino acids 21-171 of SEQ ID NO:1. The TNF-beta polypeptide can be modified to eliminate a lysine at position 28 of SEQ ID NO:1. The one or more pegylation sites can be eliminated by substitution of a lysine with a glutamic acid, valine, aspartic acid, alanine, isoleucine, or leucine. The TNF-beta polypeptide can have at least 95% identity to SEQ ID NO:1.

This document also features a polypeptide that includes a biologically active portion of a TNF-beta polypeptide, wherein the polypeptide includes amino acids 1-28 of SEQ ID NO:1, and wherein the polypeptide is modified to introduce one or more pegylation sites within amino acids 1-20 of SEQ ID NO:1. The polypeptide can be further modified to eliminate one or more pegylation sites in the biologically active portion of the TNF-beta polypeptide.

This document also features a compound that includes a TNF-beta polypeptide, wherein the TNF-beta polypeptide is modified to introduce one or more pegylation sites within amino acids 1-20 of SEQ ID NO:1, and wherein the TNF-beta polypeptide is bonded to a polyethylene glycol (PEG) moiety (e.g., wherein the TNF-beta polypeptide is modified to introduce 2, 3, or 4 pegylation sites, e.g., wherein the TNF-beta polypeptide is modified to introduce one or more lysines). The TNF-beta polypeptide can be modified to introduce two or more pegylation sites within amino acids 1-20 of SEQ ID NO:1. The TNF-beta polypeptide can be modified to include a lysine at one or both of positions 2 and 10 of SEQ ID NO:1. The lysine at one or both of positions 2 and 10 of SEQ ID NO:1 can be introduced by substitution. The TNF-beta polypeptide can be further modified to eliminate one or more pegylation sites within amino acids 21-171 of SEQ ID NO:1 (e.g., one or more lysines can be eliminated within amino acids 21-171 of SEQ ID NO:1). For example, a lysine residue can be eliminated at position 28 of SEQ ID NO:1. The one or more lysines can be eliminated by substitution with a glutamic acid, valine, aspartic acid, alanine, isoleucine, or leucine.

In another aspect, this document features a compound that includes a TNF-beta polypeptide, wherein the TNF-beta polypeptide is modified to eliminate one or more pegylation sites within amino acids 21-171 of SEQ ID NO:1, and wherein the TNF-beta polypeptide is bonded to one or more PEG moieties. The TNF-beta polypeptide can have at least 95% identity to SEQ ID NO:1. The PEG moieties can increase the circulating half life of the TNF beta polypeptide (e.g., wherein the PEG moieties increase the circulating half life of the TNF-beta polypeptide by at least 10%, 20%, 50%, 100%, 200%). The TNF-beta polypeptide can be bonded to at least two or at least three PEG moieties. The PEG moieties can have a molecular weight of about 5,000 to about 30,000 (e.g., about 10,000 or about 20,000). The TNF-beta polypeptide can be covalently bonded to the PEG moieties via a linking group (e.g., a succinimide group, an amide group, an imide group, a carbamate group, an ester group, an epoxy group, a carboxyl group, a hydroxyl group, a carbohydrate, a tyrosine group, a cysteine group, a histidine group and a combination thereof). The linking group can be a succinimide group (e.g., succinimidyl succinate, succinimidyl propionate, succinimidyl carboxymethylate, succinimidyl succinamide, N-hydroxy succinimide or a combination thereof). The succinimide group can be succinimidyl succinate, succinimidyl propionate or a combination thereof. The biological activity of the compound differs from the biological activity of an unmodified TNF-beta polypeptide by less than 20% (e.g., wherein a biological activity differs from an unmodified, non-pegylated TNF-beta polypeptide by less than 15%, 10%, or 5%, e.g., wherein the activity of the compound is substantially equivalent to the activity of a native TNF-beta polypeptide). The biological activity can be cell cytotoxicity (e.g., cytotoxicity to L929 cells) and/or suppression of cancer cell growth.

In another aspect, this document features a method of treating a cancer or a cancer symptom in a subject (e.g., a human). The method includes administering to the subject a compound that includes a TNF-beta polypeptide, wherein the TNF-beta polypeptide is modified to introduce one or more pegylation sites within amino acids 1-20 of SEQ ID NO:1, wherein the TNF-beta polypeptide is bonded to a one or more PEG moieties, and wherein the compound is administered in an amount effective to treat the cancer or the cancer symptom. The TNF-beta polypeptide can be modified to introduce two or more pegylation sites within amino acids 1-20 of SEQ ID NO:1. The TNF-beta polypeptide can be modified (e.g., by substitution) to include a lysine at one or both of positions 2 and 10 of SEQ ID NO:1. The TNF-beta polypeptide can be further modified to eliminate one or more pegylation sites (e.g., one or more lysine residues) within amino acids 21-171 of SEQ ID NO:1. For example, the TNF-beta polypeptide can be modified to eliminate a lysine at position 28 of SEQ ID NO:1. The lysines can be eliminated by substitution with a glutamic acid, valine, aspartic acid, alanine, isoleucine, or leucine. The TNF-beta polypeptide can have at least 95% identity to SEQ ID NO:1. The PEG moieties can increase the circulating half life of the TNF beta polypeptide. The compound can be administered parenterally, intravenously, intramuscularly, orally, subcutaneously, or intraperitoneally. The compound can be administered at or near a site of the cancer in the subject. The compound can be administered in a sustained release formulation. The cancer can be a kidney cancer, a breast cancer, a cancer of the gastrointestinal system (e.g., a colon cancer), a sarcoma, a lymphoma, a myeloma, prostatic cancer, a skin cancer, an esophageal cancer, a liver cancer, a pancreatic cancer, a uterine cancer, a cervical cancer, a lung cancer, a bladder cancer, or a neural cancer. The compound can be administered in an amount sufficient to reduce growth of cells of the cancer in the subject, administered in an amount sufficient to cause regression of the cancer in the subject, and/or administered in an amount that is cytotoxic to cells of the cancer. The compound can be administered daily, weekly, every other week, or monthly.

This document also features a kit for treating a cancer. The kit includes a compound that includes a TNF-beta polypeptide, wherein the TNF-beta polypeptide is modified to eliminate one or more pegylation sites within amino acids 21-171 of SEQ ID NO:1, and wherein the TNF-beta polypeptide is bonded to one or more PEG moieties, and a composition comprising an agent which is an anti-cancer agent (e.g., a chemotherapeutic drug, or an antibody that induces cytotoxicity in the cancer). The TNF-beta polypeptide can have at least 95% identity to SEQ ID NO:1.

In another aspect, this document features a method of producing a modified TNF-beta polypeptide. The method includes providing a nucleic acid encoding the TNF-beta polypeptide of SEQ ID NO:1; mutating the nucleic acid to include a sequence encoding one or more pegylation sites within amino acids 1-20 of SEQ ID NO:1; expressing the mutated nucleic acid in a cell to produce the modified TNF-beta polypeptide. The method further can include mutating the nucleic acid to eliminate one or more sequences encoding a lysine within amino acids 21-171 of SEQ ID NO:1, prior to expressing the nucleic acid. The mutated nucleic acid can be expressed in a eukaryotic cell (e.g., a yeast cell, or a mammalian cell) or prokaryotic cell (e.g., a bacteria). The method further can include conjugating the modified TNF-beta polypeptide to PEG.

In yet another aspect, this document features a method of screening a compound comprising a modified TNF-beta polypeptide. The method includes providing a modified TNF-beta polypeptide, wherein the TNF-beta polypeptide is modified to introduce one or more pegylation sites within amino acids 1-20 of SEQ ID NO:1, wherein the TNF-beta polypeptide is modified to eliminate one or more pegylation sites within amino acids 21-171 of SEQ ID NO:1, and wherein the modified TNF-beta polypeptide is bonded to one or more PEG moieties, thereby producing a compound; comparing cytotoxicity of the compound to a control, wherein cytotoxicity is compared in an assay that detects cytotoxicity to L929 cells. The method further can include selecting the compound in which the level of cytotoxicity to L929 cells is at least 30%, 40%, 50%, 75%, 85%, 95%, or 100% of the level of cytotoxicity of an unmodified TNF-beta polypeptide.

This document also features a nucleic acid encoding a TNF-beta polypeptide (e.g., a TNF-beta polypeptide having at least 95% identity to SEQ ID NO:1) wherein the TNF-beta polypeptide includes a sequence which has been modified to introduce one or more pegylation sites within amino acids 1-20 of SEQ ID NO:1. In some embodiments, the TNF-beta polypeptide has been modified to include a lysine at one or both of positions 2 and 10 of SEQ ID NO:1, and optionally, modified to eliminate a lysine at position 28 of SEQ ID NO:1.

This document also features a TNF-beta polypeptide that includes an amino acid sequence having at least 95% sequence identity to the amino acid sequence set forth in SEQ ID NO:1, wherein the polypeptide includes two or more (e.g., three or more) lysine residues within amino acids 1-20 of SEQ ID NO:1. The TNF-beta polypeptide can include a lysine at one or both of positions 2 and 10 of SEQ ID NO:1. In some embodiments, at least one lysine residue within amino acids 21 to 171 (e.g., position 28) of SEQ ID NO:1 is substituted with a different amino acid (e.g., glutamic acid, valine, aspartic acid, alanine, isoleucine, or leucine residue). The TNF-beta polypeptide can have the amino acid sequence of SEQ ID NO:2.

In another aspect, this document features a composition that includes a TNF-beta-polypeptide having at least 95% identity to the amino acid sequence set forth in SEQ ID NO:1, wherein the polypeptide includes two lysine residues within amino acids 1 to 20 of SEQ ID NO:1, and wherein the polypeptide is bonded to at least two PEG moieties (e.g., within amino acids 1-20 of SEQ ID NO:1). The TNF-beta polypeptide can include a lysine at one or both of positions 2 and 10 of SEQ ID NO:1. In some embodiments, at least one lysine residue within amino acids 21-171 (position 28) of SEQ ID NO:1 is substituted with a different amino acid (e.g., glutamic acid, valine, aspartic acid, alanine, isoleucine, or leucine). The composition has an increased circulating half life compared with a TNF-beta-polypeptide that is not bonded to the PEG moieties. The TNF-beta polypeptide can be bonded to three PEG moieties. The PEG moieties can have a molecular weight of about 5,000 to about 30,000 (e.g., about 10,000 or about 20,000). The TNF-beta polypeptide can be covalently bonded to the PEG moieties via a linking group (e.g., a succinimide group, an amide group, an imide group, a carbamate group, an ester group, an epoxy group, a carboxyl group, a hydroxyl group, a carbohydrate, a tyrosine group, a cysteine group, a histidine group and a combination thereof). For example, the linking group can be a succinimide group (e.g., succinimidyl succinate, succinimidyl propionate, succinimidyl carboxymethylate, succinimidyl succinamide, N-hydroxy succinimide or a combination thereof). The succinimide group can be succinimidyl succinate, succinimidyl propionate or a combination thereof. The biological activity of the TNF-beta polypeptide can differ from the biological activity (e.g., cell cytotoxicity or suppression of cancer cell growth) of an unmodified TNF-beta polypeptide by less than 20%.

In yet another aspect, this document features a method of treating a cancer or a cancer symptom in a subject (e.g., a human). The method includes administering to the subject a composition that includes a TNF-beta-polypeptide having at least 95% identity to the amino acid sequence set forth in SEQ ID NO:1, wherein the polypeptide includes two lysine residues within amino acids 1 to 20 of SEQ ID NO:1, and wherein the polypeptide is bonded to at least two PEG moieties (e.g., within amino acids 1-20 of SEQ ID NO:1), and wherein the compound is administered in an amount effective to treat the cancer or the cancer symptom. The composition can be administered parenterally, intravenously, intramuscularly, orally, subcutaneously, or intraperitoneally. The composition can be administered at or near a site of the cancer in the subject. The composition can be administered in a sustained release formulation. The cancer can be selected from the group consisting of a sarcoma, a lymphoma, a myeloma, prostatic cancer, a skin cancer, an esophageal cancer, a liver cancer, a pancreatic cancer, a uterine cancer, a cervical cancer, a lung cancer, a bladder cancer, a colon cancer, a kidney cancer, a breast cancer, and a neural cancer.

The details of one or more embodiments of the invention are set forth in the accompanying drawings and the description below. Other features, objects, and advantages of the invention will be apparent from the description and drawings, and from the claims. All cited patents, and patent applications and references (including references to public sequence database entries) are incorporated by reference in their entireties for all purposes.

### DESCRIPTION OF THE DRAWINGS

FIG. 1 is a graph depicting the viability of cells treated with TNF-alpha (closed circles), PEG-TNF-alpha (open circles), TNF-beta (closed triangles), and PEG-TNF-beta (open triangles) in vitro.

FIG. 2 is a graph depicting the circulating half life of TNF-alpha (closed circles), PEG-TNF-alpha (open circles), TNF-beta (closed triangles), and modified PEG-TNF-beta (open triangles).

FIG. 3 is a graph depicting the survival of mice treated with TNF-alpha, PEG-TNF-alpha, TNF-beta, and PEG-TNF-beta, at doses of 0.1 ng, 1.0 ng, and 10 ng.

FIG. 4 is a graph depicting systolic blood pressure of mice treated with TNF-alpha (closed circles), PEG-TNF-alpha (open circles), TNF-beta (closed triangles), and modified PEG-TNF-beta (P2/A10/K28 mutant)(open triangles).

Like reference symbols in the various drawings indicate like elements.

### DETAILED DESCRIPTION

The present invention is based, in part, on the discovery that TNF-beta can be modified to increase its circulating half life while preserving its biological activities. More specifically, amino acid sequence modifications described herein permit attachment of polymeric carrier molecules such as PEG, to increase the in vivo longevity of TNF-beta, wherein conjugated forms of the molecule retain potent cytotoxicity (e.g., cytotoxicity to tumor cells). Compositions containing the modified TNF-beta polypeptides conjugated to a polymer, such as PEG, exhibit increased circulating half life relative to unconjugated TNF-beta, and an increased level of biological activity.

Due to its differential binding to the p55 TNF receptor, TNF-beta produces fewer toxic side effects compared to therapy with TNF-alpha. The availability of technology to provide TNF-beta in a form that is long lived and highly active allows for less frequent dosing, reduced antigenicity, increased tumor cytotoxicity, and other beneficial effects in therapeutic treatment regimens (e.g., increased safety).

Definitions

Throughout the present disclosure, the following abbreviations may be used: PEG, polyethylene glycol; SS, succinimidyl succinate; SSA, succinimidyl succinamide; SPA, succinimidyl propionate; and NHS, N-hydroxy-succinimide.

“Polyethylene glycol” or “PEG” refers to mixtures of condensation polymers of ethylene oxide and water, in a branched or straight chain, represented by the general formula H(OCH2CH2)ₙOH, wherein n is at least 4. “Polyethylene glycol” or “PEG” is used in combination with a numeric suffix to indicate the approximate weight average molecular weight thereof. For example, PEG-5,000 (PEG5) refers to polyethylene glycol molecules having an average molecular weight of about 5,000; PEG-12,000 (PEG12) refers to polyethylene glycol molecules having an average molecular weight of about 12,000; and PEG-20,000 (PEG20) refers to polyethylene glycol molecules having an average molecular weight of about 20,000.

As used herein, the terms “individual” and “subject” refer to an animal, in some embodiments a mammal, and in some embodiments a human.

As used herein, “modulation” means either an increase (stimulation) or a decrease (inhibition) in the expression or activity of a gene or gene product.

As used herein, the term “inhibit” refers to a reduction or decrease in a quality or quantity, compared to a baseline. For example, in the context of the present invention, inhibition of cell proliferation refers to a decrease in cell proliferation as compared to baseline. In some embodiments, there is a reduction in cell proliferation of about 20%, about 40%, about 50%, about 75%, about 80%, about 85%, about 90%, about 95%, about 99%, and about 100%. Those of ordinary skill in the art can readily determine whether or not cell proliferation has been inhibited and to what extent.

As used herein, the term “biocompatible” refers to materials or compounds which are generally not injurious to biological functions and which will not result in any degree of unacceptable toxicity, including allergenic and disease states.

“Circulating half life” refers to the period of time, after injection of a composition (e.g., a composition including a modified TNF-beta polypeptide) into a patient, until a quantity of the composition has been cleared to levels one half of the original peak serum level. Circulating half life may be determined in any relevant species, including humans or mice.

As used herein, the terms “covalently bonded,” “bonded” and “coupled” are used interchangeably and refer to a covalent bond linking a polypeptide to the PEG molecule, either directly or through a linker.

As used herein, the term “therapeutically effective amount” refers to an amount of a compound effective to yield the desired therapeutic response. The specific therapeutically effective amount will, obviously, vary with such factors as the particular condition being treated, the physical condition of the patient, the type of mammal or animal being treated, the duration of the treatment, the nature of concurrent therapy (if any), and the specific formulations employed and the structure of the compounds or its derivatives. In the context of treating a cancer, the term “therapeutically effective amount” refers to an amount of a composition that reduces the growth rate of cells of a cancer, or causes stasis or regression of a cancer, or is cytotoxic to cancer cells of a subject.

As used herein, the term “prophylactically effective amount” refers to an amount of an agent effective to yield the desired prophylactic response (e.g., inhibition of tumor recurrence). The specific prophylactically effective amount will vary with such factors as the physical condition of the subject, the type of subject being treated, the duration of the treatment, the nature of concurrent therapy (if any), and the specific formulations employed and the structure of the agent.

As used herein “combination therapy” means that the individual in need of treatment is given another drug for the disease (e.g., cancer) in conjunction with a modified TNF-beta. Combination therapy can be sequential therapy where the individual is treated first with one or more drugs and then the other, or two or more drugs are given simultaneously.

As used herein, the term “sample” refers to biological material from a patient. The sample assayed by methods described herein is not limited to any particular type. Samples include, as non-limiting examples, single cells, multiple cells, tissues, tumors, biological fluids, biological molecules, or supernatants or extracts of any of the foregoing. Examples include tissue removed for biopsy, tissue removed during resection, blood, urine, lymph tissue, lymph fluid, cerebrospinal fluid, mucous, and stool samples. The sample used will vary based on the assay format, the detection method and the nature of the tumors, tissues, cells or extracts to be assayed. Methods for preparing samples are well known in the art and can be readily adapted in order to obtain a sample that is compatible with the method utilized.

Unless otherwise defined herein, scientific and technical terms used in connection with the present invention shall have the meanings that are commonly understood by those of ordinary skill in the art. Further, unless otherwise required by context, singular terms shall include pluralities and plural terms shall include the singular. Generally, nomenclatures used in connection with, and techniques of, cell and tissue culture, molecular biology, immunology, microbiology, genetics, virology and protein and nucleic acid chemistry and hybridization described herein are those well known and commonly used in the art. The methods and techniques for producing recombinant nucleic acid molecules, and for protein expression, are generally performed according to conventional methods well known in the art and as described in various general and more specific references that are cited and discussed throughout the present specification unless otherwise indicated. See, e.g., Sambrook et al. Molecular Cloning: A Laboratory Manual, 2d ed., Cold Spring Harbor Laboratory Press, Cold Spring Harbor, N.Y. (1989) and Ausubel et al., Current Protocols in Molecular Biology, Greene Publishing Associates (1992), and Harlow and Lane Antibodies: A Laboratory Manual, Cold Spring Harbor Laboratory Press, Cold Spring Harbor, N.Y. (1990), which are incorporated herein by reference. The nomenclatures used in connection with, and the laboratory procedures and techniques of, analytical chemistry, synthetic organic chemistry, and medicinal and pharmaceutical chemistry described herein are those well known and commonly used in the art. Standard techniques are used for chemical syntheses, chemical analyses, pharmaceutical preparation, formulation, and delivery, and treatment of patients.

Modified TNF-Beta

The amino acid sequence of mature human TNF-beta is shown below in Table 1. Mature human TNF-beta has 171 amino acids. The nucleic acid sequence encoding human TNF-beta is known, as are amino acid and nucleic acid sequences of TNF-beta polypeptides of other species. TNF-beta covalently linked to PEG (with or without a linking group) is referred to herein as “TNF-beta-PEG” or “pegylated TNF-beta.”

TABLE 1
Human TNF-beta amino acid sequence

(SEQ ID NO: 1)

LPGVGLTPSAAQTARQHPKMHLAHSNLKPAAHLIGDPSKQNSLLWRANTD
RAFLQDGFSLSNNSLLVPTSGIYFVYSQVVFSGKAYSPKATSSPLYLAHE
VQLFSSQYPFHVPLLSSQKMVYPGLQEPWLHSMYHGAAFQLTQGDQLSTH
TDGIPHLVLSPSTVFFGAFAL

The modified TNF-beta polypeptides provided herein include a TNF-beta amino acid sequence that has been mutated to introduce one or more polymer conjugation sites and/or delete one or more polymer conjugation sites in a manner that permits attachment of the polymer while leaving intact at least 50% (e.g., at least 60%, 70%, 80%, 90%, 95%, 97%, or 99%) of a biological activity of the polymer-conjugated polypeptide, relative to an unconjugated, unmodified form. This is achieved, at least in part, by introducing polymer conjugation sites in regions outside receptor-contacting regions of the molecule, and/or by removing polymer conjugation sites at or near receptor-contacting regions.

The p55 TNF receptor-contacting regions of human TNF-beta are found at residues 33-54, 83-85, 126-130, and 155-160 of SEQ ID NO:1 (Banner et al., Cell, 73:431-445, 1993). In various embodiments, one or more amino acids outside of one or more of these regions of SEQ ID NO:1 are modified to introduce a site for conjugation (e.g., a primary amine of a lysine residue) to a polymer (e.g., a PEG moiety). In various embodiments, the modification is a substitution of a non-lysine residue with a lysine residue. In other embodiments, the modification is an insertion of a lysine residue. The modification(s) is/are directed so as to permit attachment of the polymer in a manner that will not interfere with receptor binding and biological functions. For example, a lysine residue can be introduced within amino acids that are not implicated in receptor binding, such as amino acids 1-32, amino acids 55-82, amino acids 131-154, or amino acids 161-171 of SEQ ID NO:1. For example, the TNF-beta sequence can be modified to include a polymer conjugation site at one, two, three, four, five, or six places in the TNF-beta sequence. In some embodiments, one, two, three, or four conjugation sites (e.g., two lysine residues) are introduced within amino acids 1-32 (e.g., amino acids 1-20) of SEQ ID NO:1.

TNF-beta also can be modified to delete polymer conjugation sites, which occur in regions at or close to receptor-contacting regions of the molecule. In various embodiments, TNF-beta is modified to eliminate one of the following lysine residues of SEQ ID NO:1 (e.g., by substitution with another amino acid, such as alanine): lysine 28, lysine 39, lysine 84, lysine 89, or lysine 119. In some embodiments, a TNF-beta polypeptide is modified such that lysine residues are introduced at certain positions and other lysine residues are eliminated. An exemplary modified TNF-beta polypeptide, contains amino acid substitutions at positions 2, 10, and 28 of SEQ ID NO:1. For example, a lysine can be substituted for both the proline and the alanine at positions 2 and 10 of SEQ ID NO:1, respectively, and an alanine can be substituted for the lysine at position 28 of SEQ ID NO:1. Such a modified polypeptide is shown in the Examples below as SEQ ID NO:2.

The modified TNF-beta polypeptides described herein can include amino acid changes in addition to those that introduce or delete a polymer conjugation site. For example, TNF-beta polypeptides including naturally occurring variant residues, single amino acid substitutions, or short deletions that do not affect activity, are encompassed.

In general, a modified human TNF-beta polypeptide includes an amino acid sequence at least 90%, 95%, 97%, or 99% identical to SEQ ID NO:1. The percent identity between two amino acid sequences can be determined as follows. First, the amino acid sequences are aligned using the BLAST 2 Sequences (Bl2seq) program from the stand-alone version of BLASTZ containing BLASTP version 2.0.14. This stand-alone version of BLASTZ can be obtained from Fish & Richardson's web site (e.g., www.fr.com/blast/) or the U.S. government's National Center for Biotechnology Information web site (www.ncbi.nlm.nih.gov). Instructions explaining how to use the Bl2seq program can be found in the readme file accompanying BLASTZ. Bl2seq performs a comparison between two amino acid sequences using the BLASTP algorithm. To compare two amino acid sequences, the options of Bl2seq are set as follows: -i is set to a file containing the first amino acid sequence to be compared (e.g., C:\seq1.txt); -j is set to a file containing the second amino acid sequence to be compared (e.g., C:\seq2.txt); -p is set to blastp; -o is set to any desired file name (e.g., C:\output.txt); and all other options are left at their default setting. For example, the following command can be used to generate an output file containing a comparison between two amino acid sequences: C:\Bl2seq-ic:\seq1.txt-jc:\seq2.txt-pblastp-oc:\output.txt. If the two compared sequences share homology, then the designated output file will present those regions of homology as aligned sequences. If the two compared sequences do not share homology, then the designated output file will not present aligned sequences.

Once aligned, the number of matches is determined by counting the number of positions where an identical amino acid residue is presented in both sequences. The percent identity is determined by dividing the number of matches by the length of the amino acid sequence of SEQ ID NO:1 followed by multiplying the resulting value by 100.

It is noted that the percent identity value is rounded to the nearest tenth. For example, 78.11, 78.12, 78.13, and 78.14 is rounded down to 78.1, while 78.15, 78.16, 78.17, 78.18, and 78.19 is rounded up to 78.2. It also is noted that the length value will always be an integer.

The TNF-beta polypeptides described herein encompass non-human forms of TNF-beta (e.g., murine TNF-beta, see GenBank Accession No. CAA68529). These forms can be modified as described above for human TNF-beta, e.g., to introduce polymer conjugation sites outside regions implicated in receptor binding, and/or to delete polymer conjugation sites occurring at or near those regions.

In addition to modified TNF-beta polypeptides, the present invention provides nucleic acids encoding the polypeptides, as well as nucleic acid vectors for expression, and host cells including the nucleic acids and vectors. Suitable host cells for expression of modified TNF-beta polypeptides include, but are not limited to, mammalian (e.g., human, monkey, mouse, rabbit, hamster, etc.), fish, insect, plant, yeast (e.g., Pichia pastoris), and bacterial (e.g., E. coli) cells. Recombinant modified TNF-beta can be produced by methods such as those described in Pennica, D., et al., Nature, 312:724-729 (1981); and Streekishna, K., et al., Biochemistry, 28:4117-4125 (1989).

The biological activities and pharmacologic properties of modified TNF polypeptides can be evaluated in vitro and in vivo using methods known in the art, e.g., using L929 cells cytotoxicity assays (described in the Examples below and in Qin and Blankenstein, Cancer Res., 55:4747-4751, 1995) and established animal models for tumor treatment (e.g., human tumor xenograft models in nude mice). The biological activity of suitable modified TNF polypeptides differs by less than 20% from the biological activity of an unmodified TNF-beta polypeptide. For example, a biological activity of a modified TNF beta polypeptide can differ from an unmodified, non-pegylated TNF-beta polypeptide by less than 15%, 10%, or 5%. In some embodiments, the activity of the modified TNF-beta polypeptide is substantially equivalent to the activity of a native TNF-beta polypeptide.

Polyethylene Glycol

There are many polyethylene glycols available that differ in their molecular weight and linking group. These PEGs can have varying effects on the antigenicity, immunogenicity and circulating half life of a protein (Zalipsky, S. and Lee, C. Polyethylene Glycol Chemistry: Biotechnical and Biomedical Applications. Pp. 347-370, Plenum Press, New York, 1992; Monfardini, C., et. al., Bioconjugate Chem. 6:62-69, 1995; Delgado C; Francis G E; Fisher D. The uses and properties of PEG-linked proteins. Crit. Rev. Ther. Drug Carrier Sys., 9:249-304, 1992.)

In some embodiments, each polyethylene glycol molecule has an average molecular weight of about 10,000 to about 50,000; from about 12,000 to about 40,000, from about 15,000 to about 30,000; and about 20,000.

The polyethylene glycol may be a branched or straight chain. In some embodiments the polyethylene glycol is a straight chain. Increasing the molecular weight of the polyethylene glycol generally tends to decrease the immunogenicity of the polypeptide to which it is linked. The polyethylene glycols having the molecular weights described in the present invention may be used in conjunction with a modified TNF-beta, and, optionally, a biocompatible linking group, to treat diseases such as neoplastic diseases.

Pegylation

An modified TNF-beta may be covalently bonded to PEG via a biocompatible linking group, using methods known in the art, as described, for example, by Park et al, Anticancer Res., 1:373-376 (1981); and Zaplipsky and Lee, Polyethylene Glycol Chemistry: Biotechnical and Biomedical Applications, J. M. Harris, ed., Plenum Press, NY, Chapter 21 (1992), the disclosures of which are hereby incorporated by reference herein in their entirety.

The linking group used to covalently attach PEG to a modified TNF-beta may be any compatible linking group. In some embodiments the linking group is a biocompatible linking group. “Biocompatible” indicates that the compound or group is non-toxic and may be utilized in vitro or in vivo without causing injury, sickness, disease or death. PEG can be bonded to the linking group, for example, via an ether bond, an ester bond, a thiol bond or an amide bond. Suitable linking groups include, for example, an ester group, an amide group, an imide group, a carbamate group, a carboxyl group, a hydroxyl group, a carbohydrate, a succinimide group (including, for example, succinimidyl succinate (SS), succinimidyl propionate (SPA), succinimidyl carboxymethylate (SCM), succinimidyl succinamide (SSA) or N-hydroxy succinimide (NHS)), an epoxide group, an oxycarbonylimidazole group (including, for example, carbonyldimidazole (CDI)), a nitro phenyl group (including, for example, nitrophenyl carbonate (NPC) or trichlorophenyl carbonate (TPC)), a trysylate group, an aldehyde group, an isocyanate group, a vinylsulfone group, a tyrosine group, a cysteine group, a histidine group or a primary amine. In some embodiments the linking group is an ester group and/or a succinimide group. In some embodiments, the linking group is SS, SPA, SCM, SSA or NHS.

The particular linking groups do not appear to influence the circulating half life of a pegylated polypeptide or its biologic activity. However, if a linking group is used, in some embodiments it is important to use a biocompatible linking group. The PEG which is attached to the protein may be either a single chain, as with SS-PEG, SPA-PEG and SC-PEG, or a branched chain of PEG may be used, as with PEG2-NHS.

In some embodiments, PEG is coupled to lysine residues on a polypeptide. Alternatively, a TNF-beta polypeptide may be coupled directly to PEG (i.e., without a linking group) through an amino group, a sulfhydryl group, a hydroxyl group or a carboxyl group.

The attachment of PEG to TNF-beta increases its circulating half life (e.g., by at least 10%, 20%, 50%, 100%, 200% relative to a TNF-beta polypeptide that is not bonded to a PEG molecule). The number of PEG molecules on the polypeptide appear to be related to the circulating half life of the polypeptide. It is known that some PEG formulations are difficult to produce and yield relatively low amounts of product. Thus, to achieve an efficacious product, a balance needs to be achieved among circulating half life, antigenicity, efficiency of production, and biologic activity.

Generally, PEG is attached to a primary amine of a polypeptide. Selection of the attachment site of polyethylene glycol on the polypeptide is determined by the role of each of the sites in receptor binding of the polypeptide. From 1 to about 30 PEG molecules may be covalently bonded to a modified TNF-beta. In some embodiments, a TNF-beta polypeptide is modified with about 3 to about 10, or 7 to about 15 PEG molecules, from about 9 to about 12 PEG molecules. In some embodiments, about 30% to about 70% of the primary amino groups in the TNF-beta are modified with PEG, about 40% to about 60%, about 45% to about 55%, and about 50% of the primary amino groups in the TNF-beta are modified with PEG. Increasing the number of PEG units on TNF-beta increases its circulating half life. However, in some embodiments, increasing the number of PEG units can decrease its specific activity. Thus, in some embodiments a balance needs to be achieved between the two, as would be apparent to one skilled in the art in view of the present disclosure.

In some embodiments, the linking groups attach to a primary amine of the modified TNF-beta polypeptide via a maleimide group. Once coupled with the TNF-beta polypeptide, SS-PEG has an ester linkage next to the PEG, which may render this site sensitive to serum esterase, which may release PEG from the TNF-beta in the body. SPA-PEG and PEG2-NHS do not have an ester linkage, so they are not sensitive to serum esterase. In some embodiments, the linking group is a linking group disclosed in U.S. Pat. No. 6,737,259, which is incorporated by reference in its entirety.

Methods of Treatment

The present invention provides methods of treating cancer or a cancer symptom, or treating an individual at risk for cancer, by administering a modified TNF-beta polypeptide described herein to the individual. The methods include administering to the individual a therapeutically or prophylactically effective amount of a composition that includes the modified TNF-beta polypeptide. The modified TNF-beta polypeptide can be provided as part of a compound that includes the polypeptide covalently bonded via a linking group to polyethylene glycol (e.g., wherein each polyethylene glycol molecule has an average molecular weight of from about 10,000 to about 30,000). In some embodiments, the TNF-beta polypeptide is modified with polyethylene glycol molecules, each molecule having an average molecular weight of about 20,000. In some embodiments the linking group is selected from the group consisting of a succinimide group, an amide group, an imide group, a carbamate group, an ester group, an epoxy group, a carboxyl group, a hydroxyl group, a carbohydrate, a tyrosine group, a cysteine group, a histidine group and combinations thereof. In some embodiments the linking group is succinimidyl succinate. In some embodiments from about 7 to about 15 polyethylene glycol molecules are bonded to the TNF-beta polypeptide. In some embodiments from about 9 to about 12 polyethylene glycol molecules are bonded to the TNF-beta polypeptide.

In some embodiments the methods further include administering a therapeutically effective amount of an additional agent before, simultaneously with, or following administration of the modified TNF-beta polypeptide. For example, an anti-cancer agent such as a chemotherapeutic drug or antibody that induces cytotoxicity can be administered before, simultaneously with, or following administration of a modified TNF-beta polypeptide.

A therapeutically effective amount of one of the agents of the present invention is an amount that is effective to treat a cancer or a cancer symptom in a subject. Generally, treatment is initiated with small dosages which can be increased by small increments until the optimum effect under the circumstances is achieved. Generally, a therapeutic dosage of an agent of the present invention may be from about 0.001 to about 10 mg/kg (e.g., 0.01 to 5 mg/kg) once or twice a week to about once every two weeks. For example, the dosage may be about 0.001 mg/kg once a week as a 2 ml intramuscular injection. The compounds can be administered in one dose, continuously or intermittently throughout the course of treatment. The agent may be administered several times each day, once a day, once a week, or once every two weeks.

Methods of determining the most effective means and dosage of administration are well known to those of skill in the art. In some embodiments twice weekly dosing over a period of at least several weeks is used. Often, the modified TNF-beta will be administered for extended periods of time and may be administered for the lifetime of the individual, e.g., in order to suppress tumor growth, prevent recurrence of a tumor, or to reduce a cancer symptom. Methods of determining the most effective means and dosage of administration are well known to those of skill in the art. Single or multiple administrations can be carried out with one dose level and pattern being selected by the administrator.

The dosage administered will, of course, vary depending upon known factors, such as the pharmacodynamic characteristics of the particular agent and its mode and route of administration; the age, health and/or weight of the individual; the nature and extent of the symptoms; the kind of concurrent treatment; the frequency of treatment; the symptoms exhibited by the individual, and the effect desired.

A modified TNF-beta polypeptide may be administered in admixture with suitable pharmaceutical diluents, extenders, excipients, or carriers (collectively referred to herein as a pharmaceutically acceptable carrier) selected with respect to the intended form of administration and as consistent with conventional pharmaceutical practices. For example, in some embodiments, a composition including a modified TNF-beta polypeptide is mixed with a phosphate buffered saline solution, or any other appropriate solution known to those skilled in the art, prior to injection. The polypeptide formulation may be administered as a solid (lyophilate) or as a liquid formulation, as desired.

The compositions of the present invention are formulated according to the mode of administration to be used. In cases where pharmaceutical compositions are injectable pharmaceutical compositions, they are sterile, pyrogen free and particulate free. In some embodiments the compositions are isotonic formulations. In some embodiments additives for isotonicity can include one or more of sodium chloride, dextrose, mannitol, sorbitol and lactose. In some embodiments, the compositions are provided as isotonic solutions such as phosphate buffered saline. Stabilizers for the compositions include gelatin and albumin in some embodiments.

The modified TNF-beta compositions described herein are useful for treating cancers. Examples of cancers include, but are not limited to, breast cancer, skin cancer, bone cancer, prostate cancer, liver cancer, lung cancer (e.g., small cell lung cancer (SCLC) and non-small cell lung cancer (NSCLC) such as squamous (epidermoid) carcinoma, adenocarcinoma (including bronchoalveolar), and large-cell (undifferentiated) carcinoma), brain cancer, cancer of the larynx, gallbladder, pancreas, rectum, parathyroid, thyroid, adrenal, neural tissue, head and neck, colon, stomach, bronchi, kidneys, basal cell carcinoma, squamous cell carcinoma of both ulcerating and papillary type, metastatic skin carcinoma, osteo sarcoma, Ewing's sarcoma, veticulum cell sarcoma, myeloma, giant cell tumor, small-cell lung tumor, islet cell tumor, primary-brain tumor, acute and chronic lymphocytic and granulocytic tumors, hairy-cell tumor, adenoma, hyperplasia, medullary carcinoma, pheochromocytoma, mucosal neuromas, intestinal ganglioneuromas, hyperplastic corneal nerve tumor, marfanoid habitus tumor, Wilm's tumor, seminoma, ovarian tumor, leiomyoma tumor, cervical dysplasia and in situ carcinoma, neuroblastoma, retinoblastoma, soft tissue sarcoma, malignant carcinoid, topical skin lesion, mycosis fungoides, rhabdomyosarcoma, Kaposi's sarcoma, osteogenic and other sarcoma, malignant hypercalcemia, renal cell tumor, polycythermia vera, adenocarcinoma, glioblastoma multiforma, medulloblastoma, leukemias (e.g., acute myeloid leukemia, acute promyelocytic leukemia, acute lymphoblastic leukemia, chronic myelogenous leukemia), lymphomas (e.g., Hodgkin's disease and non-Hodgkin's lymphomas), malignant melanomas, epidermoid carcinomas, and other carcinomas and sarcomas.

The in vivo means of administration of the agents described herein will vary depending upon the intended application. As one skilled in the art will recognize, administration of a modified TNF-beta polypeptide can be carried out, for example, topically, intranasally, intraperitoneally, parenterally, intravenously, intralymphatically, intratumorly, intramuscularly, interstitially, intra-arterially, subcutaneously, intraoccularly, intrasynovial, transepithelial, mucosally and transdermally. The agents can be administered in oral dosage forms as tablets, capsules, pills, powders, granules, elixirs, tinctures, suspensions, syrups, and emulsions. The agents may also be administered in intravenous (bolus or infusion), intraperitoneal, subcutaneous, or intramuscular form, all using dosage forms well known to those of ordinary skill in the pharmaceutical arts.

Combination Therapy

Therapy with a modified TNF-beta as described herein may additionally be combined with other therapeutic agents (e.g., additional anti-cancer compounds) to provide a combination treatment regimen. In some embodiments, known anti-cancer agent may be combined with a modified TNF-beta polypeptide, as long as the combination does not eliminate the therapeutic effect of the modified TNF-beta polypeptide.

Combination therapy can be sequential (i.e., treatment with one agent first and then the second agent), or it can involve treatment with both agents at the same time. The sequential therapy can be within a reasonable time after the completion of the first therapy before beginning the second therapy. The treatment with both agents at the same time can be in the same daily dose or in separate doses. For example, in some embodiments, treatment with one agent occurs on day 1 and with the other on day 2. The exact regimen will depend on the disease or symptom being treated, the stage of disease, and the response to the treatment.

Cancer therapies for use in combination with a modified TNF-beta compound include dendritic cell therapy, therapy with chemokines, cytokines (i.e., cytokines in addition to TNF-beta, such as TNF-alpha), chemotherapeutic agents (e.g., adenosine analogs (e.g., cladribine, pentostatin), alkyl sulfanates (e.g., busulfan)), anti-tumoral antibiotics (e.g., bleomycin, dactinomycin, daunorubicin, doxorubicin, epirubicin, idarubicin, mitoxantrone, mitomycin), aziridines (e.g., thiotepa), camptothecin analogs (e.g., irinotecan, topotecan), cryptophycins (e.g., cryptophycin 52, cryptophicin 1), dolastatins (e.g., dolastatin 10, dolastatin 15), enedyine anticancer drugs (e.g., esperamicin, calichcamicin, dynemicin, neocarzinostatin, neocarzinostatin chromophore, kedarcidin, kedarcidin chromophore, C-1027 chromophore, and the like), epipodophyllotoxins (e.g., etoposide, teniposide), folate analogs (e.g., methotrexate), maytansinoids (e.g., maytansinol and maytansinol analogues), microtubule agents (e.g., docetaxel, paclitaxel, vinblastine, vincristine, vinorelbine), nitrogen mustards (e.g., chlorambucil, cyclophosphamide, estramustine, ifosfamide, mechlorethamine, melphalan), nitrosoureas (e.g., carmustine, lamustine, streptoxacin), nonclassic alkylators (e.g., altretamine, dacarbazine, procarbazine, temozolamide), platinum complexes (e.g., carboplatin, cisplatin), purine analogs (e.g., fludarabine, mercaptopurine, thioguanine), pyrimidine analogs (e.g., capecitabine, cytarabine, depocyt, floxuridine, fluorouracil, gemcitabine), substituted ureas (e.g., hydroxyurea)]; anti-angiogenic agents (e.g., canstatin, troponin I), biologic agents (e.g., ZD 1839, virulizin and interferon), antibodies and fragments thereof (e.g., anti EGFR, anti-HER-2/neu, anti-KDR, IMC-C225), anti-emetics (e.g., lorazepam, metroclopramide, and domperidone), epithelial growth factor inhibitors (e.g., transforming growth factor beta 1), anti-mucositic agents (e.g., dyclonine, lignocaine, azelastine, glutamine, corticoid steroids and allopurinol), anti-osteoclastic agents (e.g., bisphosphonates (e.g., etidronate, pamidronate, ibandronate, and osteoprotegerin)), hormone regulating agents (e.g., anti-androgens, LHRH agonists, anastrozole, tamoxifen), hematopoietic growth factors, anti-toxicity agents (e.g., amifostine), kinase inhibitors (gefitinib, imatinib), and mixtures of two or more thereof.

In some embodiments, a TNF-beta compound described herein is administered to a subject in conjunction with a cancer treatment such as a surgical procedure, radiation therapy and/or ablation therapy (e.g., laser therapy, infrared therapy and the like).

Modified TNF-beta polypeptides described herein or nucleic acids encoding such modified TNF-beta polypeptides can be combined with packaging material and sold as articles of manufacture or kits. Components and methods for producing articles of manufactures are well known. The articles of manufacture may combine one or more TNF-beta polypeptides described herein. In addition, the articles of manufacture may further include PEG, sterile water, pharmaceutical carriers, buffers, chemotherapy agents, and/or other useful reagents for treating a cancer or a cancer symptom. Instructions describing how a modified TNF-beta polypeptide is effective for treating a cancer or a cancer symptom may be included in such kits.

The invention is further described in the following examples, which do not limit the scope of the invention described in the claims.

### EXAMPLES

### Example 1

### Construction of a Modified TNF-Beta Polypeptide

A modified TNF-beta polypeptide was designed which contains mutations that permit attachment of PEG while preserving the biological activity of the polypeptide. This was achieved by introducing pegylation sites outside of regions of TNF-beta that contact the p55 TNF receptor, and deleting a pegylation site close to p55 receptor-contacting residues. The polypeptide includes these mutations: P2K, A10K, and K28A, and is referred to herein as the P2/A10/K28 mutant (numbers refer to amino acid positions within SEQ ID NO:1). The amino acid sequence of the polypeptide is shown in Table 2 (mutated positions are shown in bold, underlined font):

TABLE 2

A modified TNF-beta amino acid sequence
(P2/A10/K28 TNF-beta mutant)

(SEQ ID NO: 2)

LKGVGLTPSKAQTARQHPKMHLAHSNLAPAAHLIGDPSKQNSLLWRANTD
RAFLQDGFSLSNNSLLVPTSGIYFVYSQVVFSGKAYSPKATSSPLYLAHE
VQLFSSQYPFHVPLLSSQKMVYPGLQEPWLHSMYHGAAFQLTQGDQLSTH
TDGIPHLVLSPSTVFFGAFAL

The P2/A10/K28 TNF-beta polypeptide was coupled to PEG using the general methods described in Harras, J. M., cited above. SS-PEG, SP-PEG or NHS-PEG was added to P2/A10/K28 TNF-beta (1 mg/ml in 100 mm phosphate buffer, pH 7.2-7.5) at a 10 to 50 molar excess and mixed for one hour at room temperature. This P2/A10/K28 PEG-TNF-beta has approximately three PEG molecules being attached to the primary amines of each molecule.

Other PEG linkers and attachment sites require different pH, reaction times and amounts of PEG all of which must be empirically determined. All PEG-TNF-beta formulations were purified by removing unreacted PEG from the PEG-TNF-beta by ultra filtration using a 100 kDa cut off filter.

### Example 2

### In Vitro Activity of TNF Compounds

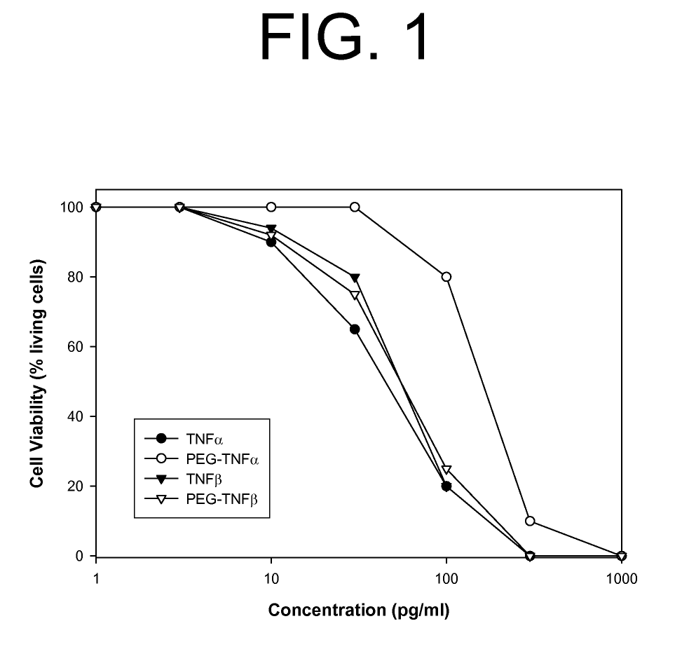

TNF-beta and PEG-TNF-beta were examined for in vitro cytotoxic activity using the L929 cytotoxicity assay (Carswell et al., Proc Natl Acad Sci U.S.A., 72(9):3666-70, 1975). Briefly, L-929 cells (ATCC) were plated in 96-well dishes and cultured for 24-48 hours. PEG-TNF-beta was added to cells at various concentrations in quadruplicate. Cell viability was determined after an overnight incubation by methyl thiazolyl tetrazolium (MTT) assays. In these experiments, the cytotoxicity of PEG-TNF-beta was compared to that of TNF-alpha, PEG-TNF-alpha, and TNF-beta. The data shown in FIG. 1 indicate that PEG-TNF-beta retains biological activity almost equivalent to that of wild type, non-pegylated TNF-beta. TNF-alpha is over 2-fold more potent than PEG-TNF-alpha.

### Example 3

### Circulating Half Life of TNF Compounds

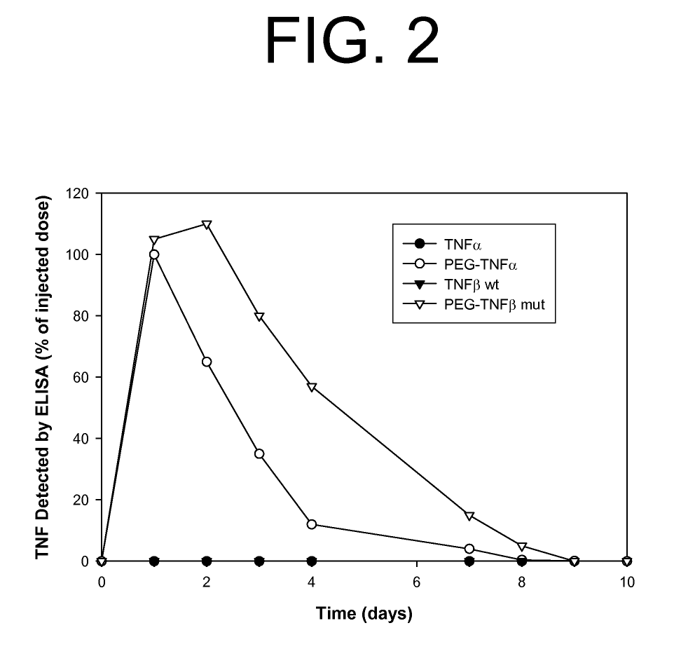

Mice (5 per group) were administered a single injection of TNF-alpha, PEG-TNF-alpha, TNF-beta, or the P2/A10/K28 PEG-TNF-beta mutant (10 ng of protein/mouse). Sera were collected from the animals prior to treatment and daily, for 10 days thereafter. The amount of TNF-alpha and TNF-beta in the sera was quantified by ELISA. As shown in FIG. 2, pegylation dramatically increased the circulating half life of P2/A10/K28 PEG-TNF-beta and TNF-alpha. Non-pegylated forms of TNF-alpha and TNF-beta were undetectable at all time points, whereas pegylated forms could be detected in sera for more than seven days after injection. Non-pegylated forms of TNF could only be detected 20 minutes after injection, at which time only 5% of the administered dose was detected, consistent with previous reports in the literature (data not shown).

### Example 4

### Tumor Cytotoxicity of TNF Compounds In Vivo

The activity of PEG-TNF-beta was tested in murine tumor models. Severe combined immunodeficient (SCID) mice were implanted with the following human kidney tumors: CRL 1933, HTB 44, or CRL 1611 (one type of tumor per was implanted in each mouse). Tumors were allowed to grow to a diameter of approximately 0.5 cm, and mice were injected intramuscular (i.m.) with TNF-alpha, PEG-TNF-alpha, TNF-beta, or PEG-TNF-beta (0.1 ng or 1 ng). The survival and tumor responses for animals with CRL 1933 tumors, HTB 44 tumors, and CRL 1611 tumors are shown in Tables 3, 4, and 5, respectively.

TABLE 3
Results From the CRL 1933 Human Kidney Cancer

| | | |
|---|---|---|
| Dose | Survival | Tumor |

| Drug | ng/mouse | 24 hr | 4 week | Response |
|---|---|---|---|---|
| TNF-alpha | 0.1 ng | 6/6 | 6/6 | 0/6 |
| | 1.0 ng | 3/6 | 2/6 | 0/6 |
| | 10 ng | 1/6 | 1/6 | 1/6 |
| PEG-TNF-alpha | 0.1 ng | 6/6 | 6/6 | 0/6 |
| | 1.0 ng | 6/6 | 6/6 | 2/6 |
| | 10 ng | 4/6 | 4/6 | 3/6 |
| TNF-beta | 0.1 ng | 6/6 | 6/6 | 0/6 |
| | 1.0 ng | 4/6 | 4/6 | 1/6 |
| | 10 ng | 1/6 | 1/6 | 1/6 |
| PEG-TNF-beta | 0.1 ng | 6/6 | 6/6 | 1/6 |
| | 1.0 ng | 6/6 | 6/6 | 4/6 |
| | 10 ng | 6/6 | 6/6 | 6/6 |

TABLE 4
Results From the HTB 44 Human Kidney Cancer

| | | |
|---|---|---|
| Dose | Survival | Tumor |

| Drug | ng/mouse | 24 hr | 4 week | Response |
|---|---|---|---|---|
| TNF-alpha | 0.1 ng | 6/6 | 6/6 | 0/6 |
| | 1.0 ng | 2/6 | 2/6 | 0/6 |
| | 10 ng | 1/6 | 1/6 | 1/6 |
| PEG-TNF-alpha | 0.1 ng | 6/6 | 6/6 | 1/6 |
| | 1.0 ng | 5/6 | 5/6 | 1/6 |
| | 10 ng | 3/6 | 3/6 | 3/6 |
| TNF-beta | 0.1 ng | 6/6 | 6/6 | 0/6 |
| | 1.0 ng | 4/6 | 3/6 | 1/6 |
| | 10 ng | 1/6 | 1/6 | 1/6 |
| PEG-TNF-beta | 0.1 ng | 6/6 | 6/6 | 2/6 |
| | 1.0 ng | 6/6 | 6/6 | 4/6 |
| | 10 ng | 5/6 | 5/6 | 5/6 |

TABLE 5
Results From the CRL 1611 Human Kidney Cancer

| | | |
|---|---|---|
| Dose | Survival | Tumor |

| Drug | ng/mouse | 24 hr | 4 week | Response |
|---|---|---|---|---|
| TNF-alpha | 0.1 ng | 6/6 | 6/6 | 0/6 |
| | 1.0 ng | 2/6 | 2/6 | 1/6 |
| | 10 ng | 1/6 | 1/6 | 1/6 |
| PEG-TNF-alpha | 0.1 ng | 6/6 | 6/6 | 0/6 |
| | 1.0 ng | 6/6 | 5/6 | 2/6 |
| | 10 ng | 5/6 | 4/6 | 2/6 |
| TNF-beta | 0.1 ng | 6/6 | 6/6 | 0/6 |
| | 1.0 ng | 5/6 | 4/6 | 1/6 |
| | 10 ng | 2/6 | 1/6 | 1/6 |
| PEG-TNF-beta | 0.1 ng | 6/6 | 6/6 | 3/6 |
| | 1.0 ng | 6/6 | 6/6 | 5/6 |
| | 10 ng | 6/6 | 6/6 | 6/6 |

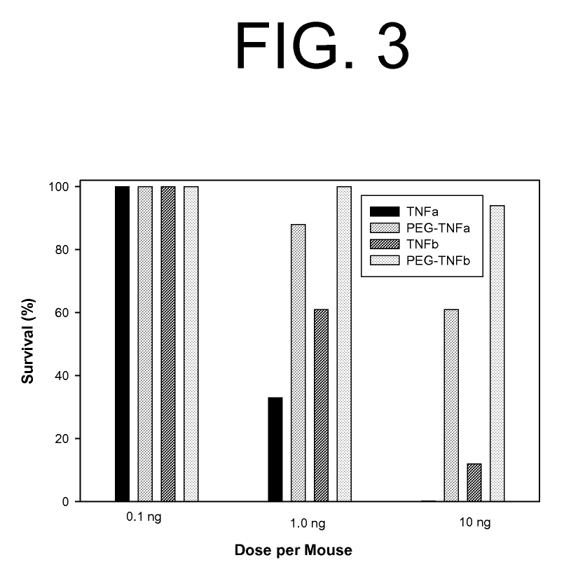

The lethality of the treatment regimens is depicted in FIG. 3. The data show that native TNF-alpha and TNF-beta were effective at killing tumors, only at doses where significant lethality was observed. Formulation of TNF-alpha and TNF-beta with PEG decreased lethality and significantly improved anti-tumor activity, as compared to non-pegylated forms of the polypeptides.

### Example 5

### Effects of TNF Compounds on Blood Pressure

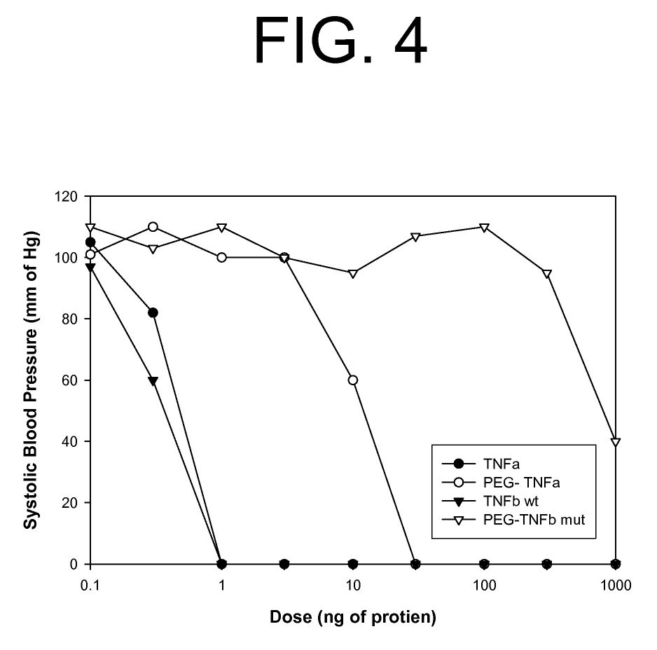

One dose limiting side effect of TNF-alpha in humans is severe hypotension, observed 10-30 minutes after treatment. To determine whether TNF-beta formulations had the same effect, systolic blood pressure was measured using a tail pressure cuff in mice administered TNF-alpha, TNF-beta and the P2/A10/K28 PEG-TNF-beta mutant. The data are shown in FIG. 4. Native TNF-alpha and TNF-beta caused hypotension at doses below those having any anti-tumor effect. Although PEG-TNF-alpha and P2/A10/K28 PEG-TNF-beta induced hypotension, they did so at doses greater than those which had anti-tumor activity. Hypotension caused by PEG-TNF-alpha was more severe than hypotension caused by P2/A10/K28 PEG-TNF-beta.

### Example 6

### Liver and Kidney Toxicity of TNF Compounds

Early human clinical testing of human TNF-alpha showed evidence of toxicity to the liver and kidney. The effects of TNF-alpha, PEG-TNF-alpha, and PEG-TNF-beta on liver and kidney function were assessed. Liver function was assessed by measuring aspartate transaminase (AST) and alanine aminotransferase (ALT). Kidney function was assessed by measuring creatinine. Organ functions were assessed by analyzing blood 48 hours after dosing with the TNF compound. Five animals per dose were analyzed. The data shown in Table 6 represent mean and SD measurements. These data show that PEG-TNF-beta is significantly less toxic than both TNF-alpha and PEG-TNF-alpha.

TABLE 6
Organ function in animals treated with TNF compounds

| Drug | Dose (ng) | AST | ALT | Creatin. |
|---|---|---|---|---|
| Control | | 156 + 66 | 93 + 16 | 0.4 + .05 |
| TNF-alpha | 1 | 114 + 123 | 173 + 93 | 0.3 + .05 |
| | 10 | 359 + 73 | 273 + 70 | 0.53 + .05 |
| PEG-TNF-alpha | 100 | 588 + 28 | 414 + 109 | 0.64 + .05 |
| | 1000 | 902 + 99 | 688 + 78 | 0.74 + .05 |
| PEG-TNF-beta | 100 | 110 + 90 | 91 + 49 | 0.4 + .05 |
| | 1000 | 101 + 93 | 99 + 7 | 0.4 + .05 |

A number of embodiments of the invention have been described. Nevertheless, it will be understood that various modifications may be made without departing from the spirit and scope of the invention. Accordingly, other embodiments are within the scope of the following claims.

What is claimed is:

1. A tumor necrosis factor-beta (TNF-beta) polypeptide comprising an amino acid sequence having at least 95% sequence identity to the amino acid sequence set forth in SEQ ID NO:1, wherein said polypeptide comprises two or more lysine residues within amino acids 1 to 20 of SEQ ID NO:1.

2. The TNF-beta polypeptide of claim 1, wherein said TNF-beta polypeptide comprises three lysine residues within amino acids 1 to 20 of SEQ ID NO:1.

3. The TNF-beta polypeptide of claim 1, wherein the TNF-beta polypeptide comprises a lysine at one or both of positions 2 and 10 of SEQ ID NO:1.

4. The TNF-beta polypeptide of claim 3, wherein at least one lysine residue within amino acids 21 to 171 of SEQ ID NO:1 is substituted with a different amino acid.

5. The TNF-beta polypeptide of claim 4, wherein the lysine residue at position 28 of SEQ ID NO:1 is substituted with a different amino acid.

6. The TNF-beta polypeptide of claim 4, wherein the different amino acid is selected from the group consisting a glutamic acid, valine, aspartic acid, alanine, isoleucine, and leucine residue.

7. The TNF-beta polypeptide of claim 1, wherein the TNF-beta polypeptide comprises the amino acid sequence of SEQ ID NO:2.

8. A composition comprising a TNF-beta-polypeptide having at least 95% identity to the amino acid sequence set forth in SEQ ID NO:1, wherein said polypeptide comprises two lysine residues within amino acids 1 to 20 of SEQ ID NO:1, and wherein said polypeptide is bonded to at least two polyethylene glycol (PEG) moieties.

9. The composition of claim 8, wherein the TNF-beta polypeptide comprises a lysine at one or both of positions 2 and 10 of SEQ ID NO:1.

10. The composition of claim 8, wherein at least one lysine residue within amino acids 21 to 171 of SEQ ID NO:1 is substituted with a different amino acid.

11. The composition of claim 10, wherein the lysine residue of position 28 of SEQ ID NO:1 is substituted with a different amino acid.

12. The composition of claim 10, wherein said different amino acid is selected from the group consisting of glutamic acid, valine, aspartic acid, alanine, isoleucine, and leucine.

13. The composition of claim 8, wherein said composition has an increased circulating half life compared with a TNF-beta-polypeptide that is not bonded to said PEG moieties

14. The composition of claim 8, wherein the TNF-beta polypeptide is bonded to three PEG moieties.

15. The composition of claim 8, wherein the PEG moieties have a molecular weight of about 5,000 to about 30,000.

16. The composition of claim 15, wherein the PEG moieties have a molecular weight of about 10,000.

17. The composition of claim 15, wherein the PEG moieties have a molecular weight of about 20,000.

18. The composition of claim 15, wherein TNF-beta polypeptide is covalently bonded to the PEG moieties via a linking group.

19. The composition of claim 18, wherein the linking group is selected from the group consisting of a succinimide group, an amide group, an imide group, a carbamate group, an ester group, an epoxy group, a carboxyl group, a hydroxyl group, a carbohydrate, a tyrosine group, a cysteine group, a histidine group and a combination thereof.

20. The composition of claim 19, wherein the linking group is a succinimide group.

21. The composition of claim 20, wherein the succinimide group is succinimidyl succinate, succinimidyl propionate, succinimidyl carboxymethylate, succinimidyl succinamide, N-hydroxy succinimide or a combination thereof.

22. The composition of claim 21, wherein the succinimide group is succinimidyl succinate, succinimidyl propionate or a combination thereof.

23. The composition of claim 8, wherein a biological activity of the TNF-beta polypeptide differs from the biological activity of an unmodified TNF-beta polypeptide by less than 20%.

24. The composition of claim 23, wherein the biological activity is cell cytotoxicity or suppression of cancer cell growth.

25. A method of treating a cancer or a cancer symptom in a subject, the method comprising:
administering to the subject a composition comprising a TNF-beta-polypeptide having at least 95% identity to the amino acid sequence set forth in SEQ ID NO:1, wherein said polypeptide comprises two lysine residues within amino acids 1 to 20 of SEQ ID NO:1, and wherein said polypeptide is bonded to at least two PEG moieties, and wherein the compound is administered in an amount effective to treat the cancer or the cancer symptom.

26. The method of claim 25, wherein the TNF-beta polypeptide comprises a lysine at one or both of positions 2 and 10 of SEQ ID NO:1.

27. The method of claim 25, wherein at least one lysine residue within amino acids 21 to 171 of SEQ ID NO:1 is substituted with a different amino acid.

28. The method of claim 27, wherein the lysine residue of position 28 of SEQ ID NO:1 is substituted with a different amino acid.

29. The method of claim 27, wherein said different amino acid is selected from the group consisting of glutamic acid, valine, aspartic acid, alanine, isoleucine, and leucine.

30. The method of claim 25, wherein the TNF-beta polypeptide is bonded to three PEG moieties.

31. The method of claim 25, wherein composition is administered parenterally, intravenously, intramuscularly, orally, subcutaneously, or intraperitoneally.

32. The method of claim 25, wherein the composition is administered at or near a site of the cancer in the subject.

33. The method of claim 25, wherein the composition is administered in a sustained release formulation.

34. The method of claim 25, wherein the subject is a human.

35. The method of claim 25, wherein the cancer is selected from the group consisting of a sarcoma, a lymphoma, a myeloma, prostatic cancer, a skin cancer, an esophageal cancer, a liver cancer, a pancreatic cancer, a uterine cancer, a cervical cancer, a lung cancer, a bladder cancer, a colon cancer, a kidney cancer, a breast cancer, and a neural cancer.


SEQUENCE LISTING (2 sequences)

SEQ ID NO: 1 - protein, 171 aa, Human
LPGVGLTPSAAQTARQHPKMHLAHSNLKPAAHLIGDPSKQNSLLWRANTDRAFLQDGFSLSNNSLLVPTSGIYFVYSQVVFSGKAYSPKATSSPLYLAHEVQLFSSQYPFHVPLLSSQKMVYPGLQEPWLHSMYHGAAFQLTQGDQLSTHTDGIPHLVLSPSTVFFGAFAL

SEQ ID NO: 2 - protein, 171 aa, Artificial Sequence
  Mutant Protein
LKGVGLTPSKAQTARQHPKMHLAHSNLAPAAHLIGDPSKQNSLLWRANTDRAFLQDGFSLSNNSLLVPTSGIYFVYSQVVFSGKAYSPKATSSPLYLAHEVQLFSSQYPFHVPLLSSQKMVYPGLQEPWLHSMYHGAAFQLTQGDQLSTHTDGIPHLVLSPSTVFFGAFAL